(19) United States
(12) Patent Application Publication
(10) Pub. No.: US 2017/0202792 A1
(43) Pub. Date: Jul. 20, 2017

(54) METHOD FOR TREATING TAU-ASSOCIATED DISEASES

(71) Applicant: NATIONAL TAIWAN NORMAL UNIVERSITY, Taipei City (TW)

(72) Inventors: Hsiu-Mei HSIEH, Taipei City (TW); Ying-Chieh SUN, Taipei City (TW); Guan-Chiun LEE, Taipei City (TW); Guey-Jen LEE-CHEN, Taipei City (TW); Ming-Tsan SU, Taipei City (TW); Hui-Chen HUANG, Taipei City (TW); Yu-Shao HSIEH, Taipei City (TW); Chia-Jen HSU, Taipei City (TW)

(21) Appl. No.: 15/238,048

(22) Filed: Aug. 16, 2016

(30) Foreign Application Priority Data
Jan. 18, 2016 (TW) 105101314

(51) Int. Cl. A61K 31/167

(52) U.S. Cl. CPC A61K 31/167

(57) ABSTRACT

A method for treating a tau-associated disease is disclosed, which comprises the step of administering a pharmaceutical composition to a subject in need. Particularly, a method for treating Alzheimer's disease is disclosed, which comprises the step of administering a pharmaceutical composition to a subject in need.

### CROSS REFERENCE TO RELATED APPLICATION

This application claims the benefits of the Taiwan Patent Application Serial Number 105101314, filed on Jan. 18, 2016, the subject matter of which is incorporated herein by reference.

### BACKGROUND OF THE INVENTION

1. Field of the Invention

The present invention relates to a method for treating tau-associated diseases, particularly, to a method for treating Alzheimer's disease (AD).

2. Description of Related Art

The probability of suffering Alzheimer's disease increases with aging. The number of patients suffering Alzheimer's disease increases due to the increasing number of the elderly population of the world and the environmental stress, including negative changes in eating habits. The reason and the mechanism of Alzheimer's disease remain unclear. Suggested mechanisms for the disorder include cholinergic hypothesis, amyloid hypothesis, and tau hypothesis. The most credible hypothesis is the abnormal tau aggregation. In this hypothesis, the imbalance between the catalytic activities of the kinase and phosphatase results in hyperphosphorylation of tau protein (Martin et al., 2013. Lessons learnt from glycogen synthase kinase 3 inhibitors development for Alzheimer's disease. Curr. Top. Med. Chem. 13, 1808-1819), and the hyperphosphorylated tau protein binds to other tau protein to form the neurofibrillary tangles which disintegrate the microtubules in the neurons. Accordingly, the delivery system in the neurons will be destroyed, resulting in the death of the neurons.

Glycogen synthase kinase-3β (GSK-3β) is involved in the formation of hyperphosphorylated tau protein and is the main kinase that phosphorylates tau protein. Hence, GSK-3β can serve as a key target for treating Alzheimer's disease by inhibiting the activity of GSK-3β for alleviating tau aggregation.

Recently, many GSK-3β inhibitors have been found and used in cell models and animal models for treating Alzheimer's disease. For example, several GSK-3β inhibitors were disclosed by Phukan (2010) (Phukan et al., 2010. GSK-3β: Role in therapeutic landscape and development of modulators. Br. J. Phajijiacol. 160, 1-19). However, none of these GSK-3β inhibitors has passed the clinical trial for clinical therapy.

Therefore, it is desirable to provide a pharmaceutical composition which is effective in treating Alzheimer's disease by inhibiting the activity of GSK-3β and preventing hyperphosphorylation of tau protein in neurons.

### SUMMARY OF THE INVENTION

In order to solve the aforementioned problems, the present invention provides a method for treating tau-associated diseases, which are caused by the hyperphosphorylation of tau protein or tau aggregation, such as Alzheimer's disease.

To achieve the object, the present invention provides a method for treating tau-associated disease, which comprises: administering a pharmaceutical composition including a compound (I) to a subject in need, wherein the compound (I) has the following formula:

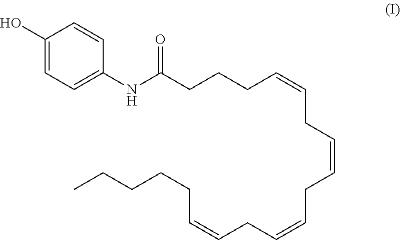 [SMILES: CCCCC/C=C\C/C=C\C/C=C\C/C=C\CCCC(=O)Nc1ccc(O)cc1]

In the present invention, the concentration of the compound (I) in the pharmaceutical composition is not particularly limited and may be adjusted based on practical usage. For example, the concentration of the compound (I) in the pharmaceutical composition may be adjusted according to the severity of the disease or other conditions, so that the pharmaceutical composition administered to the subject in need may comprise a therapeutically effective amount of the compound (I). In a preferred embodiment of the present invention, the concentration of the compound (I) may be 1 nM to 100 μM; and in another preferred embodiment of the present invention, the concentration of the compound (I) may be 10 nM to 50 μM.

In the present invention, the tau-associated diseases may comprise those neurodegenerative diseases caused by hyperphosphorylation of tau protein or tau aggregation, especially for those neurodegenerative diseases that caused by hyperphosphorylation of tau protein or tau aggregation in neurons, glial cells, or Lewy bodies. For example, those diseases may be Alzheimer's disease, frontotemporal dementia (Pick's disease), progressive supranuclear palsy, Pugilistic dementia, Lytico-Bodig disease (Parkinson dementia complex), entangled oriented dementia, argyrophilic grain dementia, ganglioglioma, gangliocytoma, subacute sclerosing panencephalitis, lead brain lesions, tuberous sclerosis complex, Hallervorden-Spatz disease, and neuronal ceroid lipofuscinosis; wherein Alzheimer's disease and frontotemporal dementia are the most common tau-associated diseases.

Another subject of the present invention is to provide a method for treating Alzheimer's disease, which comprises the step of administering a pharmaceutical composition including a compound (I), wherein the compound (I) has the following formula:

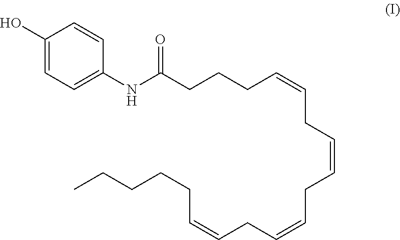 [SMILES: CCCCC/C=C\C/C=C\C/C=C\C/C=C\CCCC(=O)Nc1ccc(O)cc1]

In the present invention, the concentration of the compound (I) in the pharmaceutical composition is not particularly limited and can be adjusted based on practical usage. For example, the concentration of the compound (I) in the pharmaceutical composition may be adjusted according to the severity of the disease or other conditions, so that the pharmaceutical composition administered to the subject in need may comprise a therapeutically effective amount of the compound (I). In a preferred embodiment of the present invention, the concentration of the compound (I) in the pharmaceutical composition may be 1 nM to 100 μM; and in another preferred embodiment of the present invention, the concentration of the compound (I) of the pharmaceutical composition may be 10 nM to 50 μM.

Also, the present invention provides a method for reducing hyperphosphorylation of tau protein or tau aggregation, which comprises the step of administering a pharmaceutical composition including a compound (I), wherein the compound (I) has the following formula:

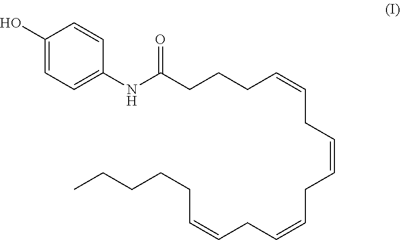 [SMILES: CCCCC/C=C\C/C=C\C/C=C\C/C=C\CCCC(=O)Nc1ccc(O)cc1]

In the present invention, the concentration of the compound (I) in the pharmaceutical composition may be 1 nM to 100 μM; and in another preferred embodiment of the present invention, the concentration of the compound (I) in the pharmaceutical composition may be 10 nM to 50 μm.

In the present invention, hyperphosphorylation of tau protein is reduced by inhibiting glycogen synthase kinase-3β (GSK-3β activity.

Furthennore, the compound (I) is N-arachidonoyl aminophenol (IUPAC: (5Z, 8Z, 11Z,14Z)-N-(4-Hydroxyphenyl)icosa-5 ,8 , 11,14-tetraen amide), which is a cannabinoid receptor agonist AM404.

In the description of the present invention, the term “reduce”, “decrease”, “ameliorate”, or “inhibit” used herein refers to the case that the pharmaceutical composition including the compound (I) of the present invention is delivered to a subject suffering from the disease caused by hyperphosphorylation of tau protein or tau aggregation, or having a tendency of developing those aforementioned diseases, in order to achieve the treatment, mitigation, slowing, or improvement of the tendency of the diseases and symptoms.

In order to implement the method according to the present invention, the above pharmaceutical composition including the compound (I) can be delivered via oral administration, parenteral administration (such as subcutaneous injection, subdural injection, intravenous injection, intramuscular injection, intrathecal injection, intraperitoneal injection, intracranial injection, intra-arterial injection, or injection at morbid site), topical administration, rectal administration, nasal administration (such as aerosols, inhalants, or powders), sublingual administration, vaginal administration, or implanted reservoir, and so on; but the present invention is not limited thereto.

Hence, the pharmaceutical composition containing the aforementioned compound (I) can be formulated into health foods or clinical drugs for preventing or treating tau-associate diseases through any medicine manufacturing procedure. Based on the requirement or usage, the pharmaceutical composition of the present invention may further comprise at least one of a pharmaceutically acceptable carrier, a diluent, or an excipient in the art.

For example, the pharmaceutical composition may be formulated into a solid form or a liquid form. When the pharmaceutical composition is formulated into a solid form, the solid excipient may comprise powders, pellets, tablets, capsules, and suppositories. The phaiiiiaceutical composition foimulated into the solid form may further comprise solid formulations, such as flavoring agents, preservatives, disintegrants, flow aids, and fillers; but the present invention is not limited thereto. In addition, the liquid excipient of the pharmaceutical composition formulated in the liquid foiin may comprise water, solution, suspension, and emulsifier; and suitable coloring agents, flavoring agents, dispersing agents, antibacterial agents, and stabilizers may also be used to prepare the liquid formulations; but the present invention is not limited thereto.

Herein, the term “therapeutically effective amount” refers to the amount of the compound (I) needed for sufficiently inducing the desired medical or pharmaceutical effects. The therapeutically effective amount may be determined by skilled person in the art (such as doctors or pharmacist) by considering various factors such as body type, age, gender, health status, the specific disease involved, the severity of the disease involved, the patient's response, the administration routes, therapy, the co-administered drugs, or other relevant conditions.

In the description of the present invention, the terms “treating” or “treatment” refer to obtaining the desired medical and physiological effects. The medical or physiological effects may refer to preventing or partially preventing a disease, preventing a disease or symptoms of the disease, curing or partially curing a disease, or a therapy for symptoms caused by a disease or adverse effects caused by the disease. The terms “treating” or “treatment” refer to treatment of the mammals, particularly of human diseases. The scope of the treatment comprises preventing a disease, namely prophylactic treatment of a patient who is susceptible to but not yet diagnosed with the disease; inhibiting a disease, that is, inhibiting or reducing the development of a disease or its clinical symptoms; or alleviating a disease, that is, alleviating a disease and/or its clinical symptoms.

### BRIEF DESCRIPTION OF THE DRAWINGS

FIG. 1 is an analysis chart showing the evaluation of inhibition of GSK-3β activity of a preferred embodiment of the present invention;

FIG. 2 is a quantification chart showing the neurite growth of cells of a preferred embodiment of the present invention;

FIG. 3a is an analysis diagram showing the expressions of HSPB1 and GRP78 of a preferred embodiment of the present invention;

FIG. 3b is a quantification chart showing the expressions of HSPB1 and GRP78 of a preferred embodiment of the present invention;

FIG. 4a is an analysis diagram showing the expressions of total GSK-3β and phosphorylated GSK-3β of a preferred embodiment of the present invention;

FIG. 4b is a quantification chart showing the expressions of total GSK-3β and phosphorylated GSK-3β of a preferred embodiment of the present invention;

FIG. 5a is an analysis diagram showing the expressions of total tau and phosphorylated tau of a preferred embodiment of the present invention;

FIG. 5b is a quantification chart showing the expressions of total tau and phosphorylated tau of a preferred embodiment of the present invention;

FIG. 6a is a diagram showing the notal bristle of the flies of a preferred embodiment of the present invention;

FIG. 6b is a quantification chart showing the number of the notal bristle of the flies of a preferred embodiment of the present invention;

FIG. 7 is a quantification chart showing the neuron numbers and neurite outgrowth in the mouse hippocampal primary culture of a preferred embodiment of the present invention;

FIG. 8 is a diagram showing the BW and BG changes of the mice of a preferred embodiment of the present invention;

FIG. 9a is an analysis diagram showing the swimming velocity of the mice in the Morris water maze (MWM) of a preferred embodiment of the present invention;

FIG. 9b is a quantification chart showing the escape latency of 4 training days in the MWM of a preferred embodiment of the present invention;

FIG. 9c is a quantification chart showing the escape latency of testing trial in the MWM of a preferred embodiment of the present invention;

FIG. 9d is a quantification chart showing the duration in target quadrant of probe trial in the MWM of a preferred embodiment of the present invention;

FIG. 10a is an analysis diagram showing the expressions of total GSK-3β, phosphorylated GSK-3β, total tau, and phosphorylated tau in hippocampal tissue of mouse of a preferred embodiment of the present invention;

FIG. 10b is a quantification chart showing the relative expressions of total GSK-3β and phosphorylated GSK-3β in hippocampal tissue of mouse of a preferred embodiment of the present invention;

FIG. 10c is a quantification chart showing the relative expressions of total tau and phosphorylated tau in hippocampal tissue of mouse of a preferred embodiment of the present invention; and

FIG. 11 is a quantification chart showing the contents of IL-6 and TNF-α in mouse serum of a preferred embodiment of the present invention.

### DETAILED DESCRIPTION OF THE PREFERRED EMBODIMENT

[Statistical Analysis]

For the following values, data are expressed as means±standard deviation (SD). More than three independent experiments were performed for each analysis, and differences between groups were evaluated using a Student's t-test. The p values were two-tailed and were considered statistically significant when p<0.05.

[Evaluation of Inhibition of GSK-3β Activity]

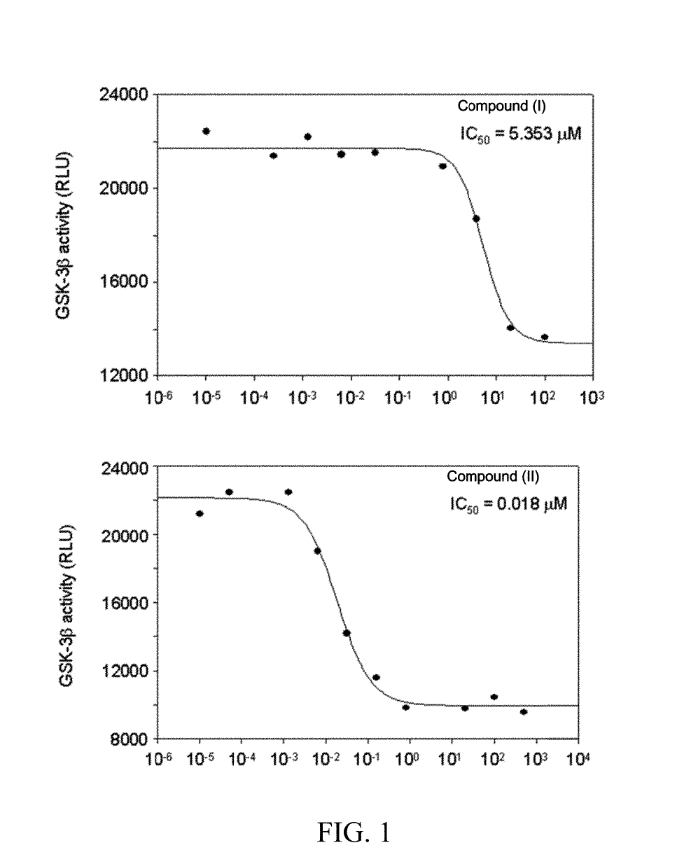

The ability of the compound (I) and SB216763 (compound (II)) for inhibiting GSK-3β activity is evaluated in the following paragraphs, wherein compound (II) is a known GSK-3β inhibitor in the art (Product No. 53442, Sigma). GSK-3β kinase activity was measured in the presence of the tested compounds (I) and (II) using ADP-Glo™ Kinase Assay system (Promega). Recombinant human GSK-3β (Product code V1991, Promega) was used as the enzyme source, and the GSK-3β substrate is derived from human muscle glycogen synthase 1 peptide (YRRAAVPPSPSLSRHSSPHQ(pS)EDEEE) which corresponds to a region of glycogen synthase that is phosphorylated by GSK-3β. Reactions were performed at 30° C. for 30 minutes in 25 μL mixture that contained 25 μM ATP, 0.2 mg/mL GSK-3β substrate, 1 ng of GSK-3β, and serial dilutions of compound (I) or compound (II). Kinase activity data were measured as relative light units (RLU) directly correlated with the amount of ADP produced and FIG. 1 showing the analysis results.

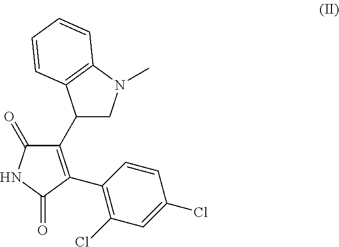 [SMILES: CN1CC(C2=C(c3ccc(Cl)cc3Cl)C(=O)NC2=O)c2ccccc21]

The IC₅₀ values of compound (I) and compound (II) were determined by using SigmaPLOT software.

Compound (II) is a known GSK-3β inhibitor in the art, the test results show that the IC₅₀ of compund (II) is 0.018 μM and the IC₅₀ of compound (I) is 5.353 μM. In addition, when the concentration of compound (I) is 0.018 μM, the residual activity of GSK-3β is 69.6±2%. According to the evaluation results that shown above, it is realized that compound (I) has the inhibition ability for GSK-3β.

[Cell Culture of SH-SY5Y tau_{RD}-DsRed]

We used SH-SY5Y human cells expressing a DsRed-tagged proaggregation mutant (ΔK280) of the C-terminal repeat domain of tau (tau_{RD}-Gln²⁴⁴-Glu³⁷² of the longest tau⁴⁴¹ isoform). The recombinant tau_{RD}-DsRed construct was under the control of a tetracycline-regulated, hybrid human cytomegalovirus (CMV)/TetO₂ promoter that can be induced by adding doxycycline. The cell lines were grown in medium containing blasticidin (5 μg/mL) and hygromycin (100 μg/mL).

[Evaluation of Neuroprotective Effects]

SH-SY5Y tau_{RD}-DsRed cells were seeded in 6-well plates (1×10⁵/well) in a medium containing all-trans retinoic acid (10 μM, Sigma). After 48 hours of incubation, cells were pre-treated with 10 μM Congo red and 10 μM compound (I) for 8 hours; after which, tau_{RD}-DsRed expression was induced with 1μg/mL doxycycline for 7 days. The cells were then fixed in 4% paraformaldehyde, permeabilized with 0.1% Triton X-100, blocked in 3% BSA, and then stained with the primary antibody anti-TUBB3 (against neuronal Class III β-tubulin) (1:1000; Covance) and with a secondary anti-rabbit Alexa Fluor® 555 antibody (1:500; Molecular Probes). Nuclei were counterstained with 4′,6-diamidino-2-phenylindole (DAPI). The total outgrowth in the untreated, Congo red-treated, and compound (I)-treated cells was assessed using MetaXpress image acquisition and analysis software.

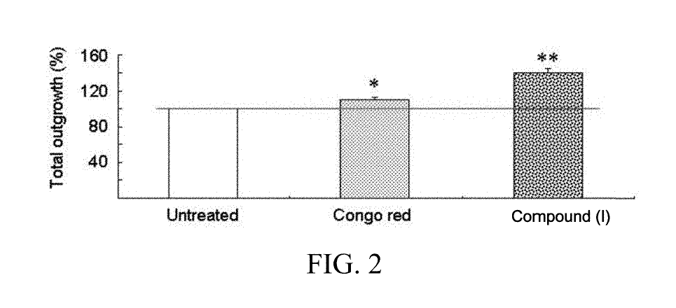

According to the fluorescence microscopy images, the quantification of the neurite growth of the untreated, Congo red-treated, and compound (I)-treated cells are shown in FIG. 2. The quantification of neurite growth of cells treated with Congo red (positive control) relative to those of untreated cells was 110% vs. 100% (p=0.026), and the quantification of neurite features of cells treated with compound (I) relative to those of untreated cells was 140% vs. 100% (p=0.005).

Furthermore, chaperones are molecules essential for proper protein folding which play a key role in protein-folding disorders in central nervous system. For example, heat shock 27 kDa protein 1 (HSPB1) is a chaperone that exerts a strong protective effect against toxicity induced by Amyloid-β(King et al., 2009. The small heat shock protein HSP27 protects cortical neurons against the toxic effects of β-amyloid peptide. J. Neurosci. Res. 87, 3161-3175), a-synuclein (Zourlidou et al., 2004. HSP27 but not HSP70 has a potent protective effect against α-synuclein-induced cell death in mammalian neuronal cells. J. Neurochem. 88, 1439-1448), and polyglutamine (Wyttenbach et al., 2002. Heat shock protein 27 prevents cellular polyglutamine toxicity and suppresses the increase of reactive oxygen species caused by huntingtin. Hum. Mol. Genet. 11, 1137-1151). Moreover, the upregulation of endoplasmic reticulum chaperones such as glucose-regulated protein, 78 kDa (GRP78) is a cellular protective response against AD (Hoshino et al., 2007. Endoplasmic reticulum chaperones inhibit the production of amyloid-(3 peptides. Biochem. J. 402, 581-589). Accordingly, the GRP78 and HSPB1 expression in tau_{RD}-DsRed SH-SYSY cells treated by Congo red and compound (I) with or without doxycycline were analyzed.

Western blotting analysis was applied for examining the expressions of HSPB1 and GRP78, the method was as follows: total proteins were extracted using RIPA buffer, which comprised 50 mM Tris-HCl, 150 mM NaCl, 1 mM EDTA, 1 mM EGTA, 0.1% SDS, 0.5% sodium deoxycholate, 1% Triton X-100, and a protease inhibitor cocktail (Calbiochem). 25 μM of total proteins were separated on 10% SDS-PAGE gels and blotted onto nitrocellulose membrane which were stained (4° C., overnight) with antibodies against DsRed (1:500; Santa Cruz), HSPB1 (1:500; Santa Cruz), and GRP78 (1:200; Santa Cruz). Next, immunoreactive bands were detected using horseradish peroxidase-conjugated goat anti-mouse, goat anti-rabbit, or donkey anti-goat IgG antibodies (1:5000; GeneTex) and chemiluminescent substrate (Millipore).

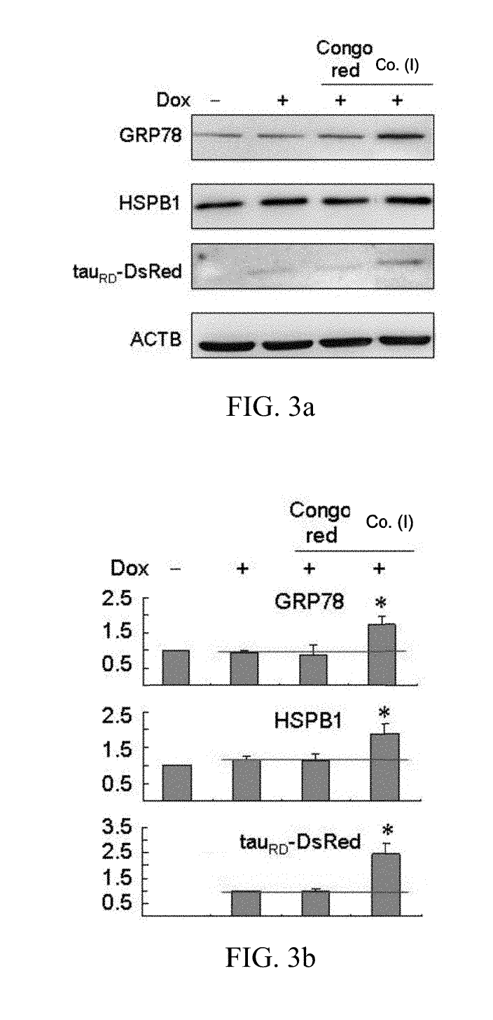

The expressions of HSPB1 and GRP78 are shown in FIG. 3a, and the quantifications thereof were shown in FIG. 3b. According to the results, the expression of HSPB1 in tau_{RD}-DsRed SH-SY5Y cells treated with compound (I) relative to that of the untreated cells were 174% vs. 100%; and the expression of GRP78 in tau_{RD}-DsRed SH-SY5Y cells treated with compound (I) relative to that of the untreated cells were 189% vs. 100%. Therefore, compound (I) led to a significant increase in the expressions of both GRP78 and HSPB1.

Based on the aforementioned test results, compound (I) may increase the neurite growth of the tau_{RD}-DsRed SH-SY5Y cells; and may increase the expressions of the chaperones HSPB1 and GRP78. It is confirmed that compound (I) exerts the neuroprotective effect.

[Evaluation of Inhibiting Hyperphosphorylation of tau Protein]

The methods of cell culture and western blot analysis are similar to that described above, except that the antibodies against GSK-3β (total and p-Ser9) (1:1000; Cell Signaling), total tau (1:500; Dako), p-tau (Ser202) (1:500; AnaSpec), p-tau (Thr231 and Ser396) (1:1000; Invitrogen), [3-actin (1:5000; Millipore), or GAPDH (1:2000; MDBio) were used herein for evaluation of the expression levels thereof in the cells.

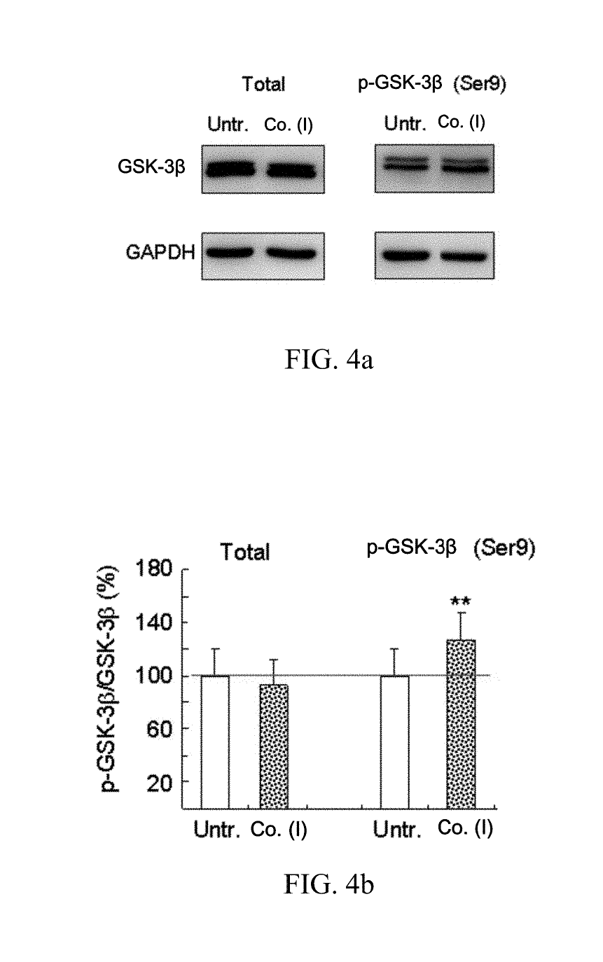

The expression levels of the total GSK-3β and phosphorylated GSK-3β were shown in FIG. 4a and the quantification thereof were shown in FIG. 4b. The analyzed results indicated that compound (I) down-regulated the GSK-3β expression in tau_{RD}-DsRed SH-SY5Y.

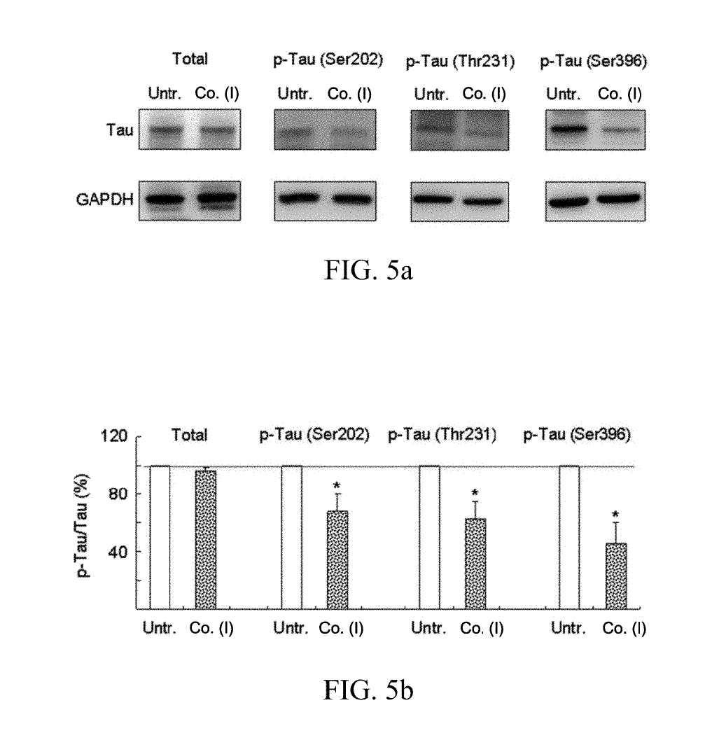

Further, the expression levels of the total tau protein and phosphorylated tau (Ser202, Thr231, and Ser396) were shown in FIG. 5a; and the quantification thereof were shown in FIG. 5b. The analyzed results indicated that the expression levels of the three tau phosphorylation sites in the cells treated with compound (I) were significantly down-regulated relative to the levels in the untreated cells. This was the case for all three tau phosphorylation sites, wherein Ser202, 32%-42% vs 100% (p=0.047-0.032); Thr231, 37%-48% vs 100% (p=0.033-0.015); and Ser396, 54%-78% vs 100% (p=0.021-0.002).

Based on the test results that described above, it is confirmed that compound (I) has the ability to decrease the phosphorylation of tau protein in tau_{RD}-DsRed SH-SY5Y cells.

Accordingly, GSK-3β activity may be inhibited while the expression level of phosphorylated GSK-3β increased in the cells treated with compound (I), which indicated that the content of phosphorylated tau protein may decreased for the reason that GSK-3β is the key for regulating the phosphorylation of tau protein (Engmann and Giese, 2009. Crosstalk between Cdk5 and GSK-3β: Implications for Alzheimer' s Disease. Front. Mol. Neurosci. 2, 2).

[In Vivo Toxicity Evaluation of tau Protein]

10 Eq-ga14 flies were treated with DMSO as the control group, and transgenic flies overexpressing tau protein driven by Eq-ga14 (Eq>tau) were treated with DMSO, 25 μM of compound (I), and 50 μM of compound (I) respectively in each group of 10 flies. The number of notal bristle of the flies was then calculated.

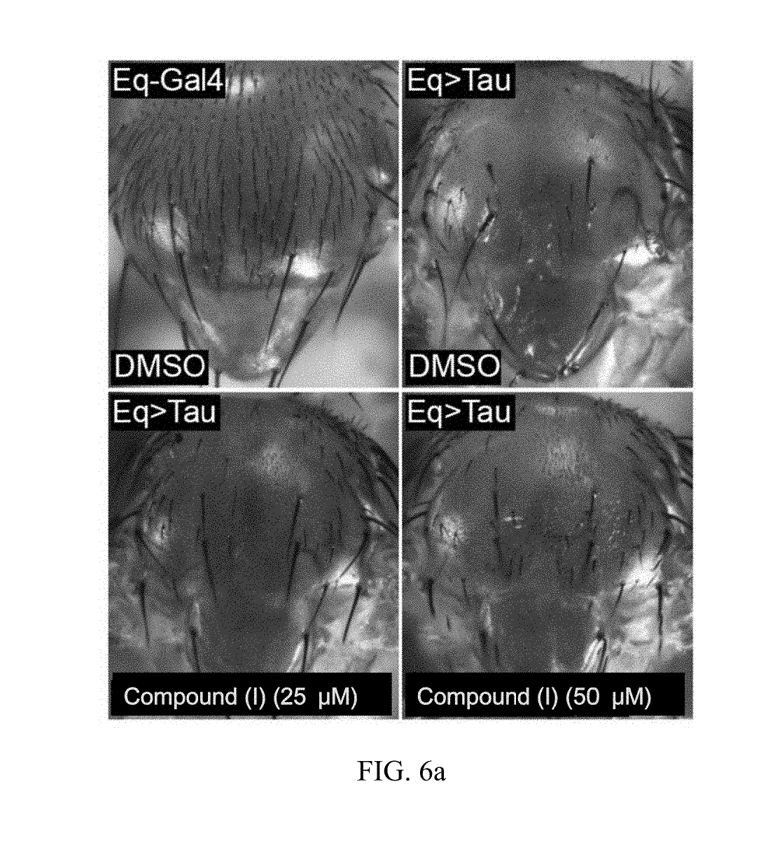

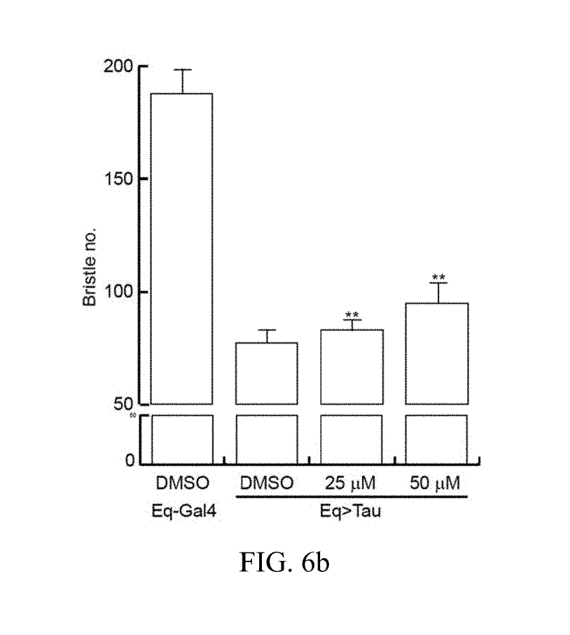

Please refer to FIG. 6a showing the notal bristle of the flies in each group, and FIG. 6b showing the number of the notal bristle (**p<0.01). The results show that the untreated Eq-ga14 flies possessed around 200 notal bristles, and the Eq-ga14 control flies treated with DMSO (control group) did not show significant effects on the growth of notal bristle. However, the transgenic flies overexpressing tau driven by Eq-gal4 (Eq>Tau) dramatically reduced the bristle number in the notum of flies when treated with DMSO, but the administration of the compound (I) effectively reduced the notal bristle loss.

According to the test results, it is confimied that compound (I) has the ability to ameliorate in vivo tau toxicity.

[Mouse Hippocampal Primary Culture Under tau Toxicity]

The mouse hippocampal primary culture cells were isolated from the hippocampi of C57BL/6J mouse embryos at days 16-18. On days in vitro (DIV) 4 and 7, 2 μM of cytosine arabinoside was added to the culture medium for reducing the glial cell populations. On DIV 9, the cells were treated with 10 nM of Wortmannin (WT) and GF109203X (GFX) to induce tau hyperphosphorylation for mimicking an AD condition. 0, 0.1, 0.25, and 0.5 μM of compound (I) were then added to the cells at DIV 9. Cells were harvested 12 hours later for immunocytochemical staining with NeuN (for neuron) and MAP2 (for neurite morphology) antibodies.

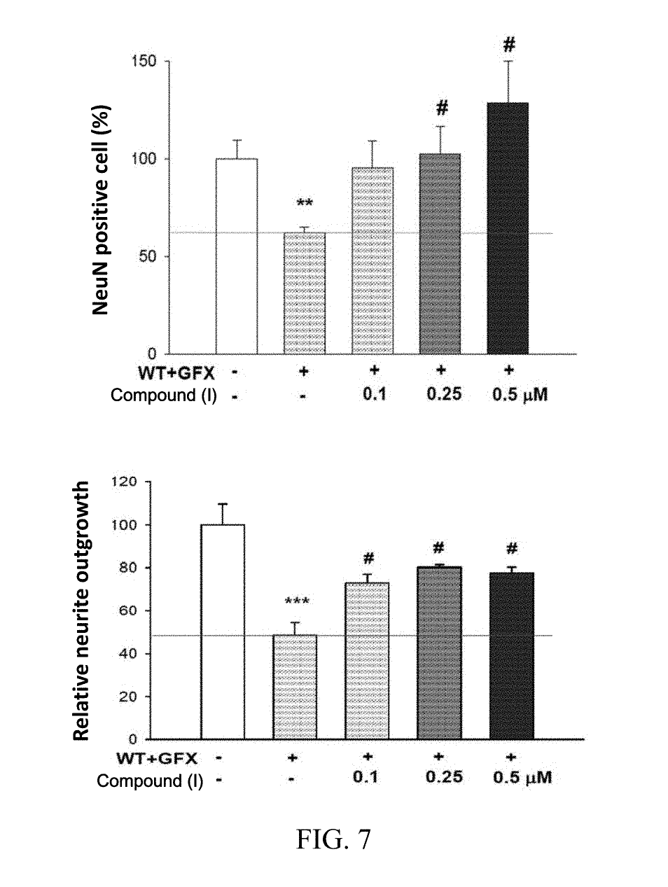

The quantification of neuron numbers and neurite outgrowth are shown in FIG. 7 (#p<0.05; **p<0.01; ***p<0.001). The results show that WT and GFX significantly reduced the neuronal survival and neurite length, which indicated that compound (I) has the ability to alleviate the phenomena of low neuronal survival rate and short neurite length induced by WT and GFX, and shows significant neuronal protective effects.

[Morris Water Maze (MWM) Test]

Hyperglycemia was induced by streptozotocin (STZ) to accelerated Alzheimer's disease progression of 6-month-old male transgenic 3×Tg−AD mice. Half of the 3×Tg−AD mice (n=30) received STZ (100 mg/kg) intraperitoneal injection at days 1, 2, 8, and 9 respectively as the high blood glucose group (HBG); another half of the 3×Tg-AD mice (n=30) received sodium citrate (0.1 M) at the same time points as the normal blood glucose group (NBG). Then, HBG and NBG were divided into two groups respectively to give four groups such as NBG-compound (I), HBG-compound (I), NBG-DMSO, and HBG-DMSO with n=15 in each group; wherein 0.25 mg/kg of compound (I) (in 30 μL) were intraperitoneal injected into those mice of NBG-compound (I) and HBG-compound (I) groups daily since day 14 for 28 days, and 30 μL of DMSO solvent were intraperitoneal injected into those mice of NBG-DMSO and HBG-DMSO groups daily since day 14 for 28 days. Both mouse body weight (BW) and blood glucose (BG) were monitored every week.

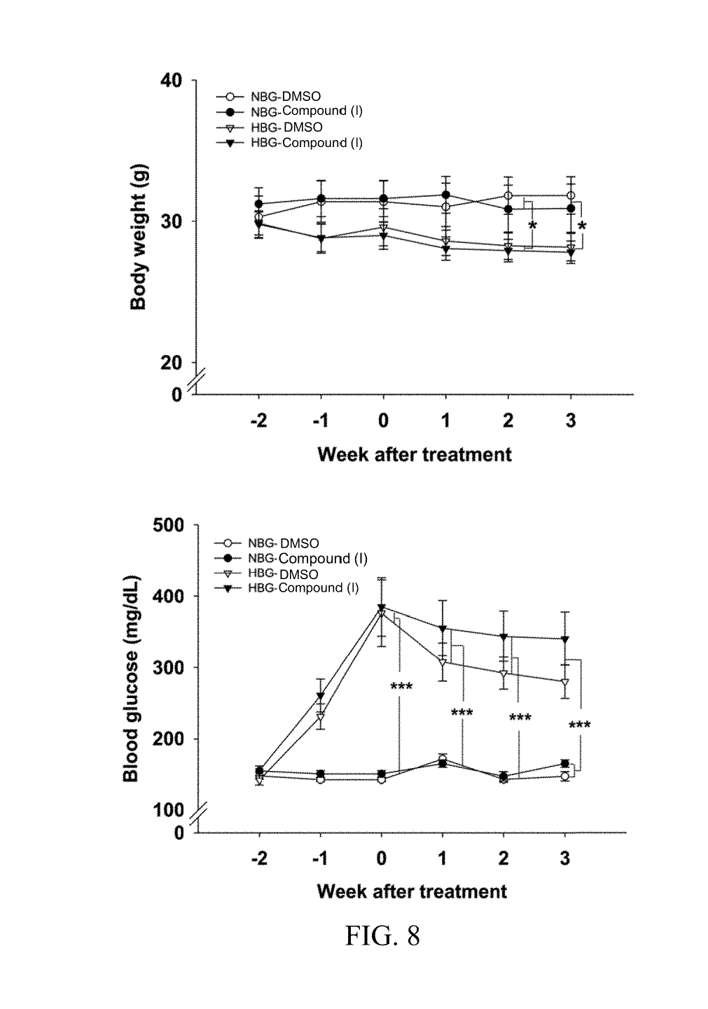

FIG. 8 shows the BW and BG changes of those mice in four groups (*p<0.05, ***p<0.001). According to the results, BW of those mice reduced after the STZ injection for 4 weeks. However, there is no significant difference in BW between the two groups treated with compound (I). On the other hand, STZ effectively raised the BG level of those mice in HBG-compound (I) group one week after injection.

Morris water maze (MWM) was conducted to evaluate the learning and memory ability of the mice at days 34-42. At first, during the 4 training days, each of the mice received four trails a day, wherein each of the mice was released into the water from a starting point that randomly varied between trials, and the time required for each of the mice to find the hidden platform to escape from the water maze (escape latency) was calculated, and the curve of 4 training days represent the learning profile of mice. 24 hours after the final training trial, the mice underwent three testing trials to determine the time required to find the hidden platform as a measure of spatial learning acquisition. The probe trials were conducted 48 hours after the end of the testing trials to evaluate the long-term spatial memory, wherein each mouse was allowed to swim freely in a pool without platform for 60 seconds, and the duration of the mouse spent in the target quadrant (where the platform was originally disposed) was measured to represent the degree of memory consolidation after learning.

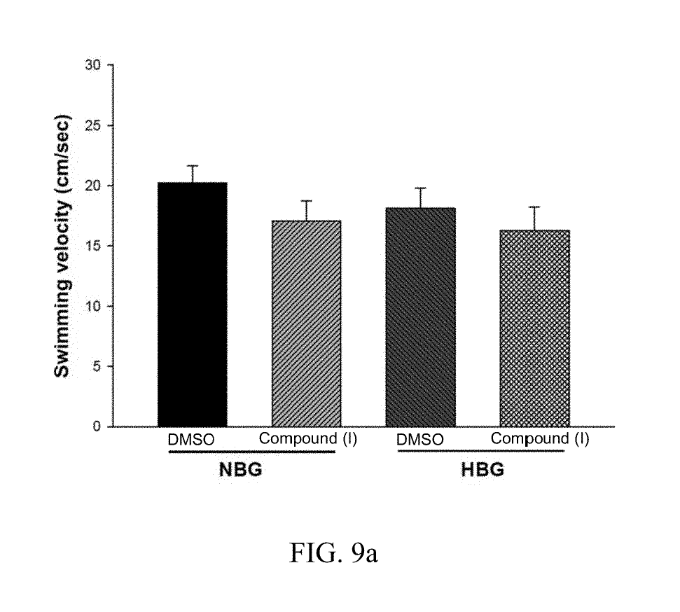

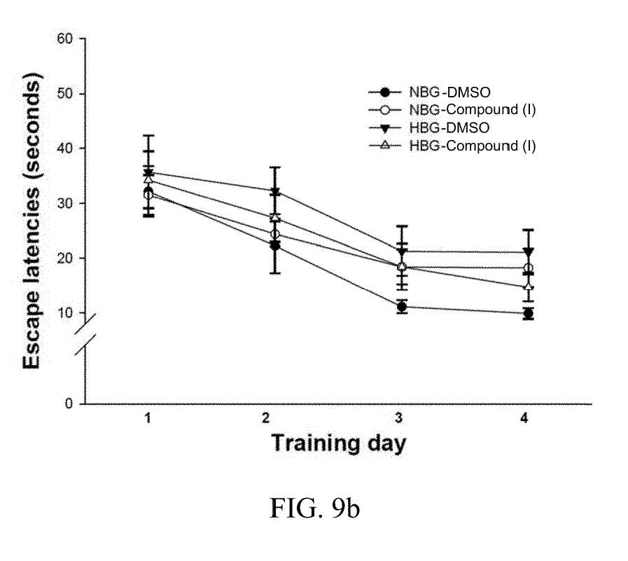

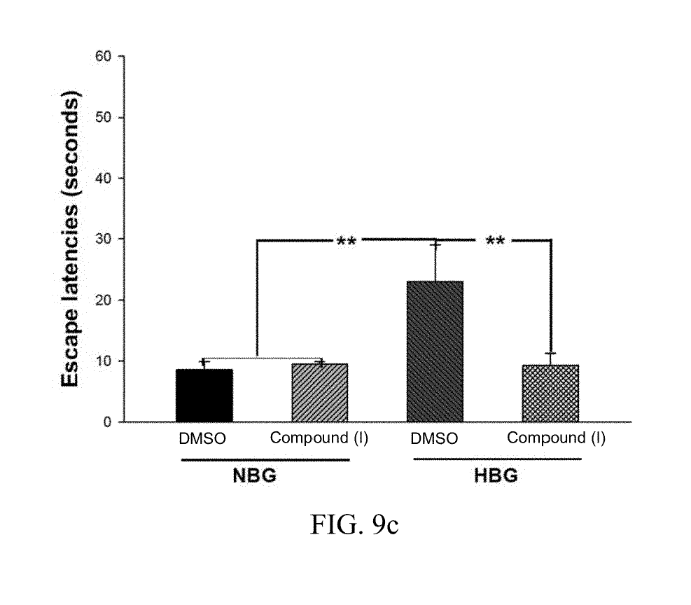

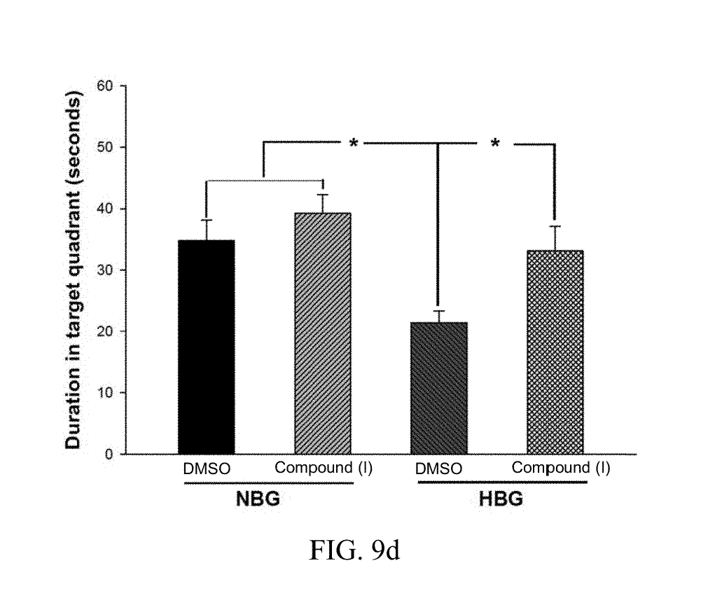

FIG. 9a illustrated the quantitative analysis diagram of the swimming velocity of during the 4 training days. As illustrated in FIG. 9a, no difference was identified in swimming velocity among 4 groups of mice. FIG. 9b illustrated the quantitative analysis diagram of the escape latency during the 4 training days, and the results show that among the groups with high blood glucose, those mice administrated with compound (I) show better learning ability than that of those mice administrated with DMSO. Furthermore, FIG. 9c illustrated the quantitative analysis diagram of the escape latency of each group (“p<0.01), wherein the duration of the mouse spent in the target quadrant, and it is proved that compound (I) can ameliorate the long-term memory damages.

[Western Blot Analysis of the Hippocampal Tissue of Mouse]

Mice were sacrificed after MWM and hippocampi were isolated for analyzing several protein expression levels by means of western blot. The protein of the hippocampi was quantified by BCA assay (Pierce), wherein 25 μg of protein was separated by SDS-PAGE and then transferred to PVDF film. Next, protein was blocked for reducing non-specific signals, and reacted with primary antibodies (GSK-3β, pS9-GSK-3β (non-activated), pT216-GSK-3β(activated), pS202Tau, pS396Tau, pT231Tau, HT7 (total Tau)), and secondary antibodies (anti-rabbit, anti-mouse IgG HRP-linked antibody; 1:10,000; Amersham Pharmacia Biotech). β-actin was used as the loading control, ECL kit was used to detect the antigen-antibody complex, and LAS-4000 (Fujifilm) was used for imaging and quantification.

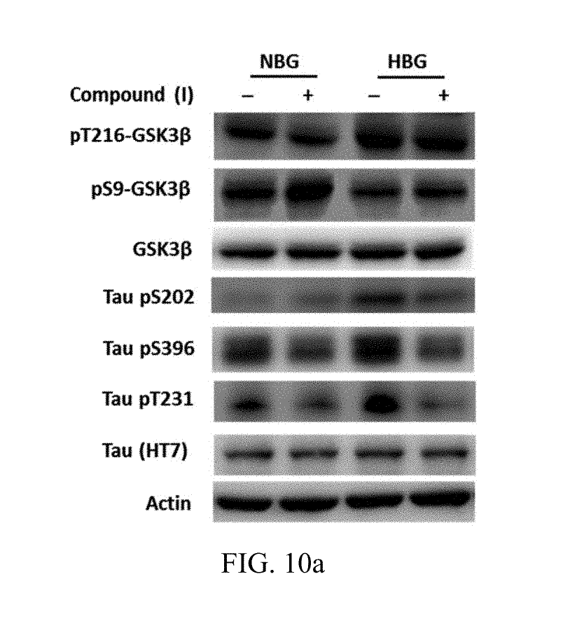

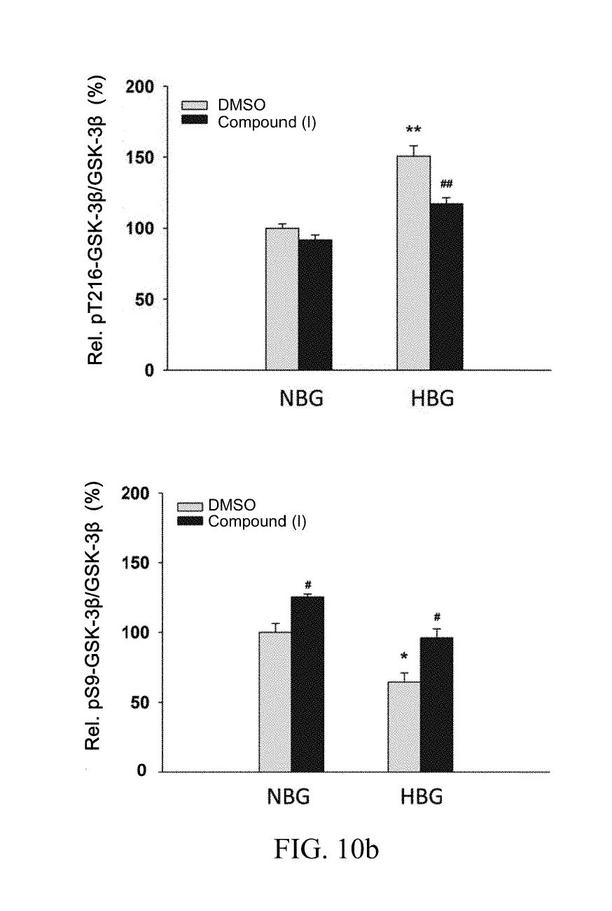

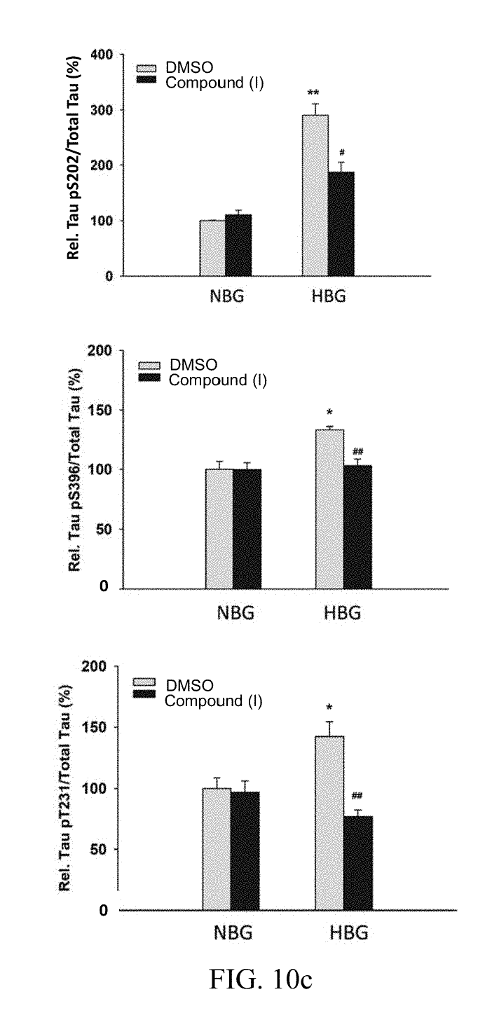

The results of the aforementioned tests were shown in FIG. 10a, and the analyzed results thereof were shown in FIG. 10b-10c, wherein *, #p<0.05; **, ##p<0.01. pT216-GSK-3β/GSK-3β and pS9-GSK-3β/GSK-3β were illustrated in FIG. 10b. It should be noted that the expression level of pT216-GSK-3β (the active form of GSK-3β) was up-regulated while the expression level of pS9GSK-3β (an inactive form of GSK-3β) was down-regulated in hyperglycemia 3×Tg-AD mice. Also, as shown in FIG. 10c, the expression level of phosphorylated tau protein was up-regulated in hyperglycemia mice; but the expression level of phosphorylated tau protein of the mice administrated with compound (I) was lower than that of the mice administrated with DMSO. Accordingly, these data indicated that compound (I) exerts inhibition for GSK-3β and reduces hyperphosphorylation of tau protein in those hyperglycemic mice.

[Immunohistochemical Staining of Aβ]

After the brain tissue of the mice was fixed and dehydrated, the tissue was cryosectioned (30 μm). The cryosections were then washed with phosphate buffered saline (PBS) for three times. After the optimal cutting temperature compound (OCT) was removed, 3% H₂O₂ was used to remove the endogenous peroxidase, and the cryosections were blocked by the blocking solution for 1 hour to reduce the non-specific antigen reactions. The primary antibodies (Aβ40, Aβ42) were added and reacted for 12 hours, the secondary antibody (1:200 dilution in blocking solution, Vecter) was added and reacted for 1 hour, and then, the avidin-biotin complex was added and reacted for 1 hour. Finally, DAB-kit was used for coloring. All the stained sections were attached to the slides, dried, dehydrated, and mounted for imaging and quantification (Image Pro Plus). The quantification results were shown in Table 1, wherein ↑, ↓:p<0.05; ↑↑, ↓↓:p<0.01.

TABLE 1

| Group | NBG-DMSO | NBG-compound (I) | HBG-DMSO | HBG-compound (I) |
|---|---|---|---|---|
| Aβ40 | 172.17 ± 7.61 | 181.88 ± 4.17 | 212.92 ± 10.49↑↑ | 164.78 ± 9.51↓↓ |
| Aβ42 | 27.50 ± 1.20 | 29.13 ± 0.64 | 33.00 ± 2.07↑ | 26.91 ± 1.28↓ |

According to the results, compound (I) treatment reduced the levels of Aβ in the hippocampus of the 3×Tg-AD mice under hyperglycemia.

[Immunohistochemical Staining of GFAP and Iba1]

After the brain tissue of the mice were fixed and dehydrated, the tissue was cryosectioned (30 μm). The cryosections were then washed with phosphate buffered saline (PBS) three times. After the optimal cutting temperature compound (OCT) was removed, 3% H₂O₂was used to remove the endogenous peroxidase, and the cryosection was blocked by the blocking solution for 1 hour to reduce non-specific antigen reactions. The primary antibodies GFAP (astrocytes) and Iba1 (microglia) were added and reacted for 12 hours, the secondary antibody (1:200 dilution in blocking solution, Vecter) was added and reacted for 1 hour, and then, the avidin-biotin complex was added and reacted for 1 hour. Finally, DAB-kit was used for coloring. All the stained sections were attached to the slides, dried, dehydrated, and mounted for imaging and quantification (Image Pro Plus) to evaluate the neuro-inflammation in mouse hippocampus. The quantification results were shown in Table 2, wherein ↑↑↑, ↓↓↓:p<0.001.

TABLE 2

| Group | NBG-DMSO | NBG-compound (I) | HBG-DMSO | HBG-compound (I) |
|---|---|---|---|---|
| GFAP | 2.50 ± 0.42 | 3.33 ± 1.02 | 43.19 ± 3.46 | 15.63 ± 2.05↓↓↓ |
| Iba1 | 36.08 ± 1.16 | 34.25 ± 2.19 | 62.53 ± 1.16↑↑↑ | 29.43 ± 0.78↓↓↓ |

According to the results, compound (I) treatment reduced the neuro-inflammation in the hippocampus of the 3×Tg-AD mice under hyperglycemia.

[Content Analysis of IL-6 and TNF-α]

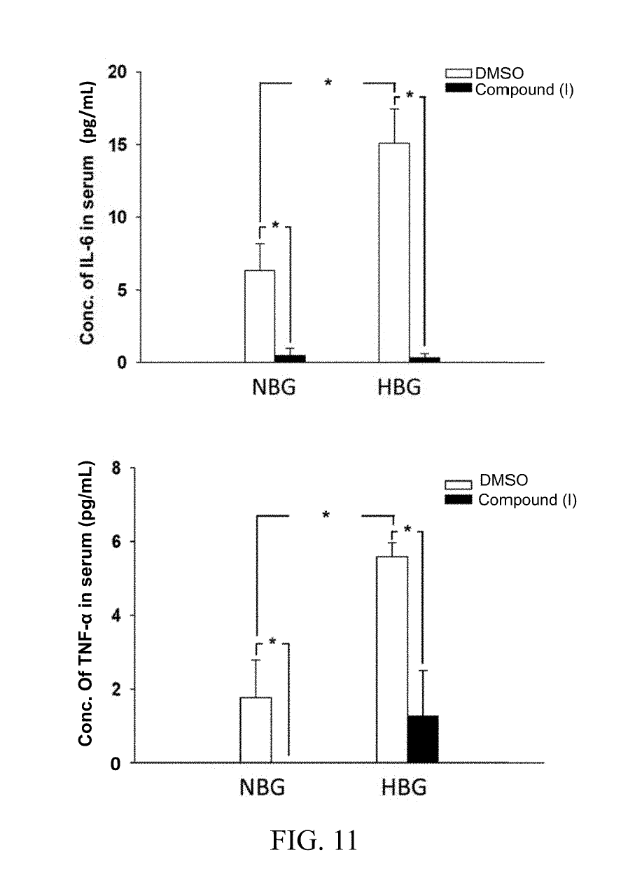

Additionally, the peripheral inflammatory cytokine in four groups of mice were evaluated. The collected blood was centrifuged (2000×g) for 20 minutes at 4° C., and the supernatant was analyzed using mouse TNF-α ELISA kit and IL-6 ELISA kit (R&D system). OD450 nm absorbance was detected by ELISA reader, and the concentration of IL-6 and of TNF-α were obtained by interpolating the standard curve and shown in FIG. 11, wherein *p<0.05. According to the results, IL-6 and TNF-α level in mice of both normal blood glucose group and high blood glucose group administrated with compound (I) were down-regulated. Therefore, it is confirmed that compound (I) has the strong anti-inflammation activity.

[Immunohistochemical Staining in Brain Region]

Neurons including the cholinergic neurons in the medial septum (MS), vertical diagonal band of Broca (VDB), and horizontal diagonal band of Broca (HDB) regions; the serotonergic neurons in the Raphe nucleus; and the noradrenergic neurons in the locus coeruleus (LC) region, which are related to cognition in the other brain regions were examined.

After the brain tissue of the mice were fixed and dehydrated, the tissue was cryosectioned (30 μm). The cryosections were then washed with phosphate buffered saline (PBS) for three times. After the optimal cutting temperature compound (OCT) was removed, 3% H₂O₂was used to remove the endogenous peroxidase, and the cryosections were blocked by the blocking solution for 1 hour to reduce non-specific antigen reactions. The primary antibodies (ChAT, TH, 5HT) were added and reacted for 12 hours, the secondary antibody (1:200 dilution in blocking solution, Vecter) was added and reacted for 1 hour, and then, the avidin-biotin complex was added and reacted for 1 hour. Finally, DAB-kit was used for coloring. All the stained sections were attached to the slides, dried, dehydrated, and mounted for imaging and quantification (Image Pro Plus).

According to the results shown in Table 3, no difference was identified for the cholinergic neurons (data not shown). However, both the serotonergic neurons in the Raphe nucleus and the noradrenergic neurons in the LC regions were significantly reduced by hyperglycemia (t: p<0.05; ↑↑↑, ↓↓↓: p<0.001). The administration of the compound (I) can effectively maintain the number of these neurons. Therefore, these results show that compound (I) has a neuroprotective effect on the AD mice.

TABLE 3

| Group | NBG-DMSO | NBG-compound (I) | HBG-DMSO | HBG-compound (I) |
|---|---|---|---|---|
| 5HT | 35.67 ± 0.92 | 31.80 ± 2.04 | 19.00 ± 1.34↓↓↓ | 33.00 ± 1.78↑↑↑ |
| TH | 75.60 ± 4.98 | 75.75 ± 3.12 | 45.79 ± 2.66↓↓↓ | 56.89 ± 4.45↓ |

According to the above evaluations, it has been proven that compound (I) of the present invention has the ability to inhibit GSK-3β activity and is effective in reducing tau aggregation, and reducing hyperphosphorylation of tau protein in cell culture model. Further, the study with Drosophila model indicates that the compound (I) can ameliorate tau toxicity. It is also confirmed that compound (I) can ameliorate the long-term memory damages, reduce hyperphosphorylation of tau protein, reduce the level of Aβ in the hippocampus, and reduce the neuroinflammation in the hippocampus of the 3×Tg-AD mice under hyperglycemia. The demonstrated effect of compound (I) in reducing tau aggregation and the level of hyperphosphorylation of tau protein suggests that it has therapeutic potential in inhibiting or reducing the tau-associated diseases, such as Alzheimer's disease, frontotemporal dementia, or other neurodegenerative disease, or its clinical symptoms, or has the effect of alleviating these diseases or its clinical symptoms.

What is claimed is:

1. A method for treating tau-associated diseases, comprising:
administering a pharmaceutical composition including a compound (I) to a subject in need, wherein the compound (I) has the following formula:

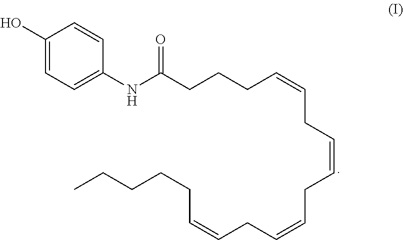 [SMILES: CCCCC/C=C\C/C=C\C/C=C\C/C=C\CCCC(=O)Nc1ccc(O)cc1]

2. The method as claimed in claim 1, wherein a concentration of the compound (I) is 1 nM to 100μM.

3. The method as claimed in claim 1, wherein a concentration of the compound (I) is 10 nM to 50 μM.

4. The method as claimed in claim 1, wherein the tau-associated disease is a neurodegenerative disease caused by hyperphosphorylation of tau protein or tau aggregation.

5. The method as claimed in claim 1, wherein the tau-associated disease is a neurodegenerative disease caused by hyperphosphorylation of tau protein or tau aggregation in neurons, glial cells, or Lewy bodies.

6. The method as claimed in claim 1, wherein the tau-associated disease is Alzheimer's disease or frontotemporal dementia.

7. A method for treating Alzheimer's disease, comprising:
administering a pharmaceutical composition including a compound (I) to a subject in need, wherein the compound (I) has the following formula:

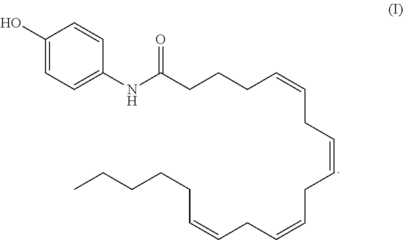 [SMILES: CCCCC/C=C\C/C=C\C/C=C\C/C=C\CCCC(=O)Nc1ccc(O)cc1]

8. The method as claimed in claim 7, wherein a concentration of the compound (I) is 1 nM to 100 μM.

9. The method as claimed in claim 7, wherein a concentration of the compound (I) is 10 nM to 50 μM.

10. A method for reducing hyperphosphorylation of tau protein or tau aggregation, comprising: administering a pharmaceutical composition including a compound (I) to a subject in need, wherein the compound (I) has the following formula:

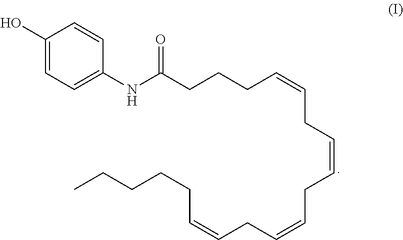 [SMILES: CCCCC/C=C\C/C=C\C/C=C\C/C=C\CCCC(=O)Nc1ccc(O)cc1]

11. The method as claimed in claim 10, wherein a concentration of the compound (I) is 1 nM to 100 μM.

12. The method as claimed in claim 10, wherein a concentration of the compound (I) is 10 nM to 50 μM.

13. The method as claimed in claim 10, wherein the hyperphosphorylation of tau protein is reduced by inhibiting glycogen synthase kinase-3β (GSK-3β) activity.
